(19) United States
(12) Patent Application Publication
(10) Pub. No.: US 2023/0128799 A1
(43) Pub. Date: Apr. 27, 2023

(54) PROCESS FOR BLEACHING KERATIN FIBERS USING AN OXIDIZING COMPOSITION AND UV-VISIBLE RADIATION

(71) Applicant: L'Oreal, Paris (FR)

(72) Inventors: Gabin VIC, Clichy (FR); Fabrice OSOLIN, SAINT-OUEN (FR); Valentin LUX, SAINT-OUEN (FR); Frédéric WOODLAND, SAINT-OUEN (FR)

(21) Appl. No.: 17/911,968

(22) PCT Filed: Mar. 17, 2021
(86) PCT No.: PCT/EP2021/056841

§ 371 Date: Sep. 15, 2022

(30) Foreign Application Priority Data
Mar. 17, 2020 (FR) 2002598

(51) Int. Cl. A61K 8/40; A61K 8/22; A61Q 5/08; A61Q 5/06

(52) U.S. Cl. CPC A61K 8/40; A61K 8/22; A61Q 5/08; A61Q 5/065; A61K 2800/81; A61K 2800/4322

(57) ABSTRACT

The present invention relates to a process for bleaching keratin fibers, in particular human keratin fibers such as the hair, comprising a step of applying, to said fibers, an oxidizing composition comprising hydrogen peroxide and a non-peroxygenated ammonium salt with a molar mass of greater than 40 g/mol and a step of irradiating said fibers with UV-visible radiation having a wavelength ranging from 200 to 800 nm and a fluence ranging from 1 to 5000 J/cm2, after application of said oxidizing composition.

The present invention relates to a process for bleaching keratin fibers, in particular human keratin fibers such as the hair, comprising a step of applying an oxidizing composition to said fibers followed by a step of irradiating said fibers with UV-visible radiation having a wavelength ranging from 200 to 800 nm and a fluence ranging from 1 to 5000 J/cm².

The invention relates to the technical field of hair bleaching.

The bleaching or lightening of keratin fibers, in particular human keratin fibers such as the hair, is performed by oxidation of the “melanin” pigment resulting in the dissolution and partial or total removal of this pigment.

Processes for lightening or bleaching human keratin fibers generally consist in applying an aqueous composition comprising at least one oxidizing agent, under alkaline pH conditions in the vast majority of cases. The role of this oxidizing agent is in particular to degrade the melanin of the keratin fibers, which, depending on the nature of the oxidizing agent present, leads to more or less pronounced lightening of the fibers. Thus, for relatively mild lightening, the oxidizing agent is generally hydrogen peroxide. When more pronounced lightening is desired, use is usually made of peroxygenated salts, for instance persulfates, in the presence of hydrogen peroxide.

The lightening processes described in the prior art usually have the drawback of comprising steps of which the implementation times prove to be quite long. For example, a lightening process may comprise a step of leaving in the oxidizing composition which may reach up to 50 minutes, to which it is generally necessary to add the required time linked to the application of said composition. In other words, the application times and the leave-in times for the compositions used during such lightening processes prove to be quite considerable, which can make their use tiresome for the user and/or the hairstyler.

Such lightening processes also have the disadvantage of using compositions which have quite high concentrations of active substances, such as of alkaline agents and/or of oxidizing agents, which can make these processes expensive.

Furthermore, standard lightening processes have the drawback of modifying the natural shade of the keratin fibers during bleaching thereof, which usually results in the appearance of an unattractive orangey background. For this reason, it is sought to develop lightening processes which are friendlier to the natural shades of keratin fibers.

Moreover, bleaching processes which use a light source are already known from the prior art.

Indeed, U.S. Pat. No. 4,792,341 describes a bleaching process comprising a step of irradiation carried out by means of a laser or a flash lamp.

Patent application WO 91/06279 describes, for its part, a photochemical bleaching process using a composition containing from 0.5% to 5% by weight of a photosensitizer and a composition which can release a hydrogen radical.

Patent application WO 2007/048473 describes a dyeing or bleaching process using a step of irradiation by means of UV radiation having a wavelength ranging from 200 to 600 nm or with particular irradiation devices.

These documents do not describe effective bleaching processes which use a step of irradiation with UV-visible radiation having a particular fluence range.

Patent application WO 2015/165949 describes a bleaching or dyeing process using a composition comprising a chemical oxidizing agent and a step of irradiation with UV-visible radiation of improved effectiveness.

However, there is a need to further improve the existing bleaching processes.

There is therefore a real need to develop processes for bleaching keratin fibers, in particular human keratin fibers such as the hair, which do not have the drawbacks mentioned above, i.e. which are faster to carry out and which are capable of being kind to the natural shades and the integrity of the keratin fibers.

This aim is achieved by the present invention, of which a subject is in particular a process for bleaching keratin fibers, in particular human keratin fibers such as the hair, comprising:

i) a step of applying, to said fibers, an oxidizing composition comprising:
at least one non-peroxygenated ammonium salt with a molar mass of greater than 40 g/mol;
hydrogen peroxide; and
optionally at least one alkaline agent different from the non-peroxygenated ammonium salt with a molar mass of greater than 40 g/mol;

the oxidizing composition having a pH greater than or equal to 7.5,

ii) a step of irradiating said fibers with UV-visible radiation having a wavelength ranging from 200 to 800 nm and a fluence ranging from 1 to 5000 J/cm², after application of said composition.

In other words, the process according to the invention successfully uses a step of applying a composition having a pH greater than or equal to 7.5 containing one or more non-peroxygenated ammonium salts with a molar mass of greater than 40 g/mol, hydrogen peroxide and optionally an alkaline agent different from the non-peroxygenated ammonium salt with a molar mass of greater than 40 g/mol, and a step of irradiating the fibers as described above.

The irradiation step of the bleaching process according to the invention makes it possible to advantageously reduce the leave-in time needed for the oxidizing composition to effectively lighten the keratin fibers, or even to eliminate it. The total treatment time may then correspond to the application time of the oxidizing composition on the keratin fibers and to the irradiation time of the fibers.

It results therefrom that the process according to the invention makes it possible to reduce the keratin fiber treatment time, thereby making it easier to implement.

Advantageously, the irradiation step is carried out by means of a light-emitting source which makes the process according to the invention more efficient.

Furthermore, the process according to the invention leads to improved lightening performance levels while being capable of being kinder to the natural shade of the keratin fibers than the lightening processes of the prior art. Indeed, it is noted that the keratin fibers treated with the lightening process according to the invention do not exhibit a lightening background that is as orangey as the fibers treated with the lightening processes of the prior art.

In other words, the lightening process according to the invention makes it possible to produce tints or shades that are more natural and esthetic.

Other features and advantages of the invention will emerge more clearly on reading the description and the examples that follow.

In the text hereinbelow, and unless otherwise indicated, the limits of a range of values are included within that range.

The expression “at least one” is equivalent to the expression “one or more”.

The human keratin fibers treated via the process according to the invention are preferably the hair.

As indicated previously, the bleaching process according to the invention comprises a step i) of applying an oxidizing composition to the keratin fibers.

Oxidizing Composition

The oxidizing composition comprises hydrogen peroxide.

The hydrogen peroxide may be present in the oxidizing composition in a total content ranging from 0.5% to 12% by weight, preferably from 1% to 10% by weight and more preferably from 3% to 9% by weight, relative to the total weight of the oxidizing composition.

The oxidizing composition also comprises at least one non-peroxygenated ammonium salt with a molar mass of greater than 40 g/mol.

The term “ammonium salt” means any mineral or organic compound comprising in its structure at least one ammonium-based cation and an anion derived from a mineral or organic acid.

The term “non-peroxygenated ammonium salt” means any ammonium salt which does not comprise an —O—O— bond.

The non-peroxygenated ammonium salt is different particular from ammonium percarbonate, perborate or persulfate.

The ammonium salt is different from ammonium hydroxide.

The ammonium salt may be a mineral ammonium salt or an organic ammonium salt.

The term “mineral salt” means a salt that does not comprise more than one carbon atom in its structure.

The mineral ammonium salt may be acid, alkaline or neutral.

The term “acid salt” means a salt which, at 5% in water, results, at 25° C., in a pH of less than 6.5.

The term “alkaline salt” means a salt which, at 5% in water, results, at 25° C., in a pH of greater than 7.5.

The term “neutral salt” means a salt which, at 5% in water, results, at 25° C., in a pH ranging from 6.5 to 7.5.

The non-peroxygenated mineral ammonium salts with a molar mass of greater than 40 g/mol may be chosen from ammonium carbonate, ammonium bicarbonate, ammonium carbamate, ammonium chloride, ammonium sulfate, ammonium hydrogen sulfate, ammonium nitrate, ammonium phosphate, ammonium monohydrogen phosphate, ammonium dihydrogen phosphate.

The organic ammonium salts may be chosen from ammonium lactate, ammonium acetate.

Preferably, the non-peroxygenated ammonium salt with a molar mass of greater than 40 g/mol is a mineral ammonium salt.

According to a first variant, the non-peroxygenated ammonium salt is chosen from acid non-peroxygenated mineral ammonium salts.

The acid non-peroxygenated ammonium salt is preferably chosen from ammonium chloride and ammonium sulfate. According to this first variant, the acid mineral ammonium salt is preferably ammonium chloride.

According to a second variant, the non-peroxygenated ammonium salt is chosen from alkaline non-peroxygenated mineral ammonium salts. The alkaline mineral ammonium salt is preferably chosen from ammonium carbonate, ammonium bicarbonate, ammonium carbamate, ammonium phosphate. According to this second variant, the alkaline mineral salt is ammonium carbonate.

Preferably, the non-peroxygenated ammonium salt with a molar mass of greater than 40 g/mol is chosen from ammonium chloride, ammonium carbonate and ammonium bicarbonate.

The non-peroxygenated ammonium salt(s) may be present in the oxidizing composition in a total amount ranging from 0.1% to 20%, preferably from 0.5% to 15% and more preferably from 1% to 10% by weight relative to the total weight of the oxidizing composition.

The oxidizing composition may also comprise an alkaline agent different from the non-peroxygenated ammonium salt with a molar mass of greater than 40 g/mol.

The term “alkaline agent” means any agent for increasing the pH of the composition in which it is present.

It may be mineral or organic.

More particularly, the alkaline agent(s) that can be used in the oxidizing composition of the invention may be chosen from:

a) aqueous ammonia,

b) organic amines, for instance alkanolamines such as mono-, di- and triethanolamines, isopropanolamine and 2-amino-2-methyl-1-propanol, and also derivatives thereof,

c) oxyethylenated and/or oxypropylenated ethylenediamines,

d) mineral or organic hydroxides,

e) alkali metal silicates such as sodium metasilicates,

f) amino acids, preferably basic amino acids, such as arginine, glycine, lysine, ornithine, citrulline and histidine, or the alkali metal or alkaline-earth metal salts thereof,

g) carbonates and bicarbonates, particularly of a primary, secondary or tertiary amine, or of an alkali metal or alkaline-earth metal, and

h) the compounds of formula (III) below:

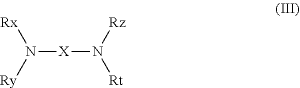

in which X is a linear or branched C₁-C₆ alkylene residue, optionally substituted with one or more hydroxyl or C₁-C₆, better still C₁-C₄, alkyl groups; Rx, Ry, Rz and Rt, which may be identical or different, represent a hydrogen atom or a C₁-C₆ alkyl, C₁-C₆ hydroxyalkyl or C₁-C₆ aminoalkyl group.

Examples of such compounds of formula (III) that may be mentioned include 1,3-diaminopropane and 1,3-diamino-2-propanol.

The mineral or organic hydroxides are preferably chosen from hydroxides of an alkali metal, hydroxides of an alkaline-earth metal, for instance sodium hydroxide or potassium hydroxide, hydroxides of a transition metal, such as hydroxides of metals from groups III, IV, V and VI of the Periodic Table of the Elements, hydroxides of lanthanides or actinides and guanidinium hydroxide.

The hydroxide may be formed in situ, for instance guanidine hydroxide, formed by reacting calcium hydroxide and guanidine carbonate.

The preferred alkaline agents are chosen in particular from aqueous ammonia, organic amines, in particular alkanolamines such as monoethanolamine, triethanolamine and 2-amino-2-methyl-1-propanol, alkali metal or alkaline earth metal hydroxides, sodium carbonate and sodium bicarbonate, alkali metal or alkaline-earth metal (meta)silicates, in particular sodium (meta)silicates, arginine, sodium glycinate or potassium glycinate and mixtures thereof.

Preferably, the alkaline agent is chosen from alkali metal or alkaline-earth metal (meta)silicates. More preferably, the alkaline agent is chosen from sodium (meta)silicates.

The alkaline agent(s), if they are present, represent a total content ranging from 0.1% to 20% by weight, preferably from 0.2% to 15% by weight and more preferably from 0.3% to 10% by weight, relative to the total weight of the oxidizing composition.

According to a variant, the oxidizing composition comprises hydrogen peroxide and at least one alkaline non-peroxygenated mineral ammonium salt, preferably chosen from ammonium carbonates and bicarbonates, and more preferably chosen from ammonium carbonates.

According to this variant, the oxidizing composition does not necessarily comprise an alkaline agent different from the non-peroxygenated ammonium salt with a molar mass of greater than 40 g/mol.

Preferably, the oxidizing composition comprises less than 5% by weight of peroxygenated salts, preferably less than 3% of peroxygenated salts, more preferably less than 1% of peroxygenated salts, relative to the total weight of the composition. More preferably, the oxidizing composition does not comprise any peroxygenated salts.

The oxidizing composition may comprise one or more hair dyes, preferably chosen from direct dyes and oxidation dyes.

The oxidizing composition is aqueous.

The oxidizing composition comprises water in an amount ranging from 20% to 70%, preferably from 30% to 60% by weight, relative to the total weight of the oxidizing composition.

The pH of the oxidizing composition is preferably between 7.5 and 12, preferably between 8 and 11, better still between 9 and 10.5.

The oxidizing composition may be in various forms, for instance a solution, an emulsion or a gel.

The oxidizing composition can be obtained by mixing at least two compositions.

In particular, the oxidizing composition may result from the mixing of a composition (A) comprising at least one non-peroxygenated ammonium salt with a molar mass of greater than 40 g/mol and optionally at least one alkaline agent different from the non-peroxygenated ammonium salt and of a composition (B) comprising hydrogen peroxide, and optionally of a third composition comprising at least one alkaline agent different from the non-peroxygenated ammonium salt with a molar mass of greater than 40 g/mol.

Composition (A) is preferably anhydrous.

For the purposes of the present invention, the term “anhydrous composition” means a composition having a water content of less than 5% by weight, preferably less than 2% by weight, and/or a composition which does not contain any added water, that is to say that the water which may be present in the composition according to the invention is more particularly bound water, such as the water of crystallization of salts, or traces of water absorbed by the starting materials used in the production of the compositions according to the invention.

Composition (B) is preferably aqueous.

The oxidizing composition, composition (A) and composition (B) may also comprise one or more adjuvants, such as solvents, fatty substances, anionic, cationic, nonionic, amphoteric or zwitterionic polymers or mixtures thereof, anionic, nonionic, cationic and/or amphoteric surfactants, mineral thickeners, and in particular fillers such as clays or talc, organic thickeners with, in particular, anionic, cationic, nonionic and amphoteric polymeric associative thickeners, pigments, antioxidants, penetrants, sequestrants, fragrances, dispersants, film-forming agents, ceramides, preserving agents, opacifiers, antidandruff agents, agents for preventing hair loss.

Irradiation Step

As previously indicated, the process according to the invention comprises a step ii) of irradiating the keratin fibers by means of UV-visible radiation having a wavelength ranging from 200 to 800 nm and a fluence ranging from 1 to 5000 J/cm², after application of said composition.

Preferably, 90% of the energy of the radiation emitted by the irradiation source is between 200 and 800 nm. More preferentially, 95% of the energy of the radiation emitted by the irradiation source is between 200 and 800 nm.

In one preferred variant of the invention, the light source emits “monochromatic” radiation, i.e. radiation centered on a given wavelength with a very low dispersion of the energy emitted outside a range of ±10 nanometers relative to said wavelength.

Preferably, the UV-visible radiation has a wavelength ranging from 280 to 700 nm and particularly ranging from 350 to 500 nm.

More particularly, the UV-visible radiation has a wavelength of 385 nm, 405 nm or 455 nm (±10 nanometers).

The fluence of the UV-visible radiation ranges from 1 to 5000 J/cm², preferably from 1 to 2000 J/cm², particularly from 10 to 1000 J/cm², more particularly from 20 to 800 J/cm².

Advantageously, the irradiation step of the process according to the invention is carried out using one or more light sources, preferably one or more light-emitting sources.

More advantageously, the UV-visible radiation is emitted by one or more light-emitting sources made up of one or more light-emitting elements such as light-emitting diodes (LEDs) or organic light-emitting diodes (OLEDs).

The light-emitting source can be chosen from light-emitting diodes (LEDs) or organic light-emitting diodes (OLEDs).

In other words, the irradiation step is carried out by means of one or more light-emitting sources, thereby making it possible to efficiently reduce the treatment time of the process.

Preferably, the irradiation source may be composed of one or more light-emitting diodes.

The diodes further have the advantage of being easier to miniaturize and more energetic. The diodes also make it possible to reduce the irradiation time for a given fluence.

In particular, the irradiation source used in the process according to the invention is borne by tongs as described in French patent application no. 1903781 that can be moved along a lock of hair.

The UV-visible radiation may be continuous or may be pulsed at a pulse frequency ranging from 0.001 to 1000 Hz and preferably ranging from 0.01 to 100 Hz.

In one variant of the invention, the irradiance of the radiation emitted by the source(s) varies from 1 to 50 Watt/cm² on the substrate treated.

Preferably, the keratin fibers, once treated by the oxidizing composition, are irradiated for a time of less than 60 minutes. In particular, the irradiation time varies from 1 to 60 minutes, preferably more 1 to 30 minutes, more preferably from 1 to 20 minutes, in particular for a time ranging from 1 to 5 minutes.

The irradiation source may be moved along a lock of hair one or more consecutive times.

Preferably, an accessory such as a film or a wrapper can be placed on the keratin fibers after the application of the oxidizing composition on the keratin fibers, and before the irradiation step.

The accessory may make it possible to enclose the keratin fibers on which the oxidizing composition was applied.

Preferably, the accessory is transparent to the irradiation wavelength.

Preferably, the leave-in time of the oxidizing composition on the keratin fibers before the irradiation step may have a duration of less than 30 minutes, preferably less than 10 minutes, more preferably less than 5 minutes.

Preferably, the irradiation step is carried out immediately after the application of the oxidizing composition on the keratin fibers.

The expression “immediately after” is understood to mean less than 5 minutes after the end of the application of the composition or the placement of an accessory such as a film or a wrapper on the keratin fibers. In other words, a leave-in time of the oxidizing composition on the keratin fibers is not necessary.

Preferably, the process according to the invention also comprises a step of rinsing out the oxidizing composition after the irradiation step.

According to one variant of the invention, the oxidizing composition is applied to a part of the surface of the keratin fibers, then, in a second step, said partial or total surface is subsequently irradiated with UV-visible radiation as previously defined.

In accordance with another variant of the invention, the oxidizing composition is applied to the entire surface of the keratin fibers, then, in a second step, the entire surface is irradiated with UV-visible radiation as previously defined.

According to a third variant of the invention, the oxidizing composition is applied to the entire surface of the keratin fibers, then, in a second step, a part of said surface is irradiated (part of the surface which may be predefined) with UV-visible radiation as previously defined.

Such an embodiment allows the creation of lightened patterns on the surface of the keratin fibers.

The invention also relates to a process for bleaching keratin fibers, in particular human keratin fibers such as the hair, comprising:
the mixing of a first composition (A) comprising at least one non-peroxygenated ammonium salt with a molar mass of greater than 40 g/mol and optionally at least one alkaline agent different from the non-peroxygenated ammonium salt with a molar mass of greater than 40 g/mol with a second composition (B) comprising hydrogen peroxide and optionally with a third composition comprising at least one alkaline agent different from the non-peroxygenated ammonium salt with a molar mass of greater than 40 g/mol, in order to obtain an oxidizing composition having a pH greater than or equal to 7.5; then
the application of the oxidizing composition to said fibers; then
the irradiation of said fibers with UV-visible radiation having a wavelength ranging from 200 to 800 nm and a fluence ranging from 1 to 5000 J/cm².

The examples that follow serve to illustrate the invention without, however, being limiting in nature.

In the following examples, the keratin fibers used are hair having a tone level equal to 4 (TL4), which corresponds to a lock of chestnut brown hair.

The notion of “tone level” is based on the classification of natural shades, one tone separating each shade from the shade immediately following or preceding it. This definition and the classification of natural shades are well known to hairstyling professionals and are published in the book “Sciences des traitements capillaires [Hair treatment science]” by Charles Zviak, 1988, published by Masson, pp. 215 and 278.

The tone levels range from 1 (black) to 10 (very light blond), one unit corresponding to one tone; the higher the figure, the lighter the shade.

### EXAMPLES

### Example 1

A. Compositions Tested

The following compositions were prepared from the ingredients in the tables below, the amounts of which are expressed as weight percentages of active material:

TABLE 1

| | A1 | A2 | A3 | A4 | A5 | A6 | A7 | A8 | A9 |
|---|---|---|---|---|---|---|---|---|---|
| Ammonium | 10.8 | — | — | 10.8 | 10.8 | — | 10.8 | 14.6 | — |
| chloride | | | | | | | | | |
| Ammonium | — | 9.76 | — | — | — | — | — | — | — |
| carbonate | | | | | | | | | |
| Ammonium | — | — | 15.97 | — | — | — | — | — | — |
| bicarbonate | | | | | | | | | |
| Ammonium | — | — | — | — | — | 15.6 | — | — | — |
| acetate | | | | | | | | | |
| Ammonium | — | — | — | — | — | | — | — | 21.6 |
| lactate | | | | | | | | | |
| Sodium | 37 | 37 | 37 | 37 | 37 | 37 | 37 | 37 | 37 |
| silicate | | | | | | | | | |
| Purified | 15.2 | 15.2 | 15.2 | — | — | 15.2 | | — | 15.2 |
| smectite | | | | | | | | | |
| Kaolin | — | — | — | — | 15.2 | — | 15.2 | — | — |
| Ammonium | — | — | | 15.2 | — | — | — | | — |
| sulfate | | | | | | | | | |
| Sodium | — | — | — | — | — | — | | 11.4 | — |
| metasilicate | | | | | | | | | |
| Mineral oil | 3 | 3 | 3 | 3 | 3 | 3 | 3 | 3 | 3 |
| Sodium | 34 | 35.04 | 28.83 | 34 | 34 | 29.20 | — | 34 | 23.2 |
| chloride | | | | | | | | | |
| Potassium | — | — | — | — | — | — | 34 | — | — |
| chloride | | | | | | | | | |

These compositions in powder form are mixed with the aqueous composition below, comprising hydrogen peroxide, in the ratio 1+3 (A+B).

TABLE 2

| | Composition B |
|---|---|
| Hydrogen peroxide | 9.0 |
| Sclerotium gum | 2.5 |
| Tetrasodium etidronate | 0.06 |
| Tetrasodium pyrophosphate | 0.04 |
| Sodium salicylate | 0.035 |
| Phosphoric acid | qs pH 2.2 |
| Water | qs 100 |

B. Light-Emitting Source

A blue LED bank constructed so as to be composed of several blue light-emitting diodes (LEDs), the wavelength of the blue light of which is centered on 405 nm, is used.

The instrument is composed of sufficient light-emitting diodes appropriately installed to illuminate a surface area of 18 cm². The source delivers a total fluence between 1 and 5000 J/cm². The irradiation is carried out on one part of a lock of hair, with periodic movement of the light source so as to give the same fluence over the entire lock of hair.

C. Procedure

The various mixtures obtained are applied to locks of hair having a TL4 tone level, in a proportion of 10 grams of mixture per 1 gram of lock of hair, and they are enclosed in a wrapper that is transparent to the irradiation wavelength.

Each lock of hair is irradiated at a fluence of 80 J/cm² and at a wavelength of 405 nanometers for a duration of 160 seconds. The light source is moved periodically along the lock so as to never illuminate the entire lock in one go.

The lock is rinsed and then washed with shampoo.

D. Results

The intensity L* of the color of the locks is measured with a Minolta Spectrophotometer CM2600D colorimeter.

The variation in the color intensity ΔL of the locks before and after treatment with the process according to the invention is calculated according to the following equation: ΔL=L*−L₀*, L* representing the values measured after treatment and L₀* representing the values measured before treatment.

The larger the value of ΔL, the greater the difference in color of the lock before and after treatment, and in the present case, the more bleached the lock is.

The results are reported in table 1 below.

TABLE 3

| | A1 + B | A2 + B | A3 + B | A4 + B | A5 + B | A6 + B | A7 + B | A8 + B | A9 + B |
|---|---|---|---|---|---|---|---|---|---|
| pH of the | 9.2 | 9.8 | 9.3 | 9.1 | 9.5 | 9.8 | 9.5 | 9.7 | 9.6 |
| mixture | | | | | | | | | |
| ΔL | 25.9 | 18.15 | 17.45 | 22.45 | 25.72 | 25.27 | 26.48 | 30.53 | 22.03 |

It is observed that the process according to the invention enables effective bleaching of the locks of hair.

### Example 2

A. Compositions Tested

The following oxidizing compositions are prepared, the amounts of which are expressed as weight percentages of active material.

TABLE 4

| | Oxidizing | Oxidizing |
|---|---|---|
| | composition C | composition C1 |
| | (invention) | (comparative) |
| Hydrogen peroxide | 7.2 | 7.2 |
| Ammonium chloride | 1.07 | — |
| Sodium silicate | 0.45 | — |
| Ammonium hydroxide | — | 0.7 |
| Water | qs 100 | qs 100 |
| pH | 9.5 | 9.5 |

The source of ammonium is in an equimolar amount in the two compositions.

B. Light-Emitting Source

The same light-emitting source is used as in example 1.

C. Procedure

The compositions (C) and (C1) are applied respectively to locks of hair having a TL4 tone level, in a proportion of 10 grams per 1 gram of lock of hair, and they are enclosed in a wrapper that is transparent to the irradiation wavelength.

Each lock of hair is irradiated at a fluence of 80 J/cm² and at a wavelength of 405 nanometers for a duration of 160 seconds. The light source is moved periodically along the lock so as to never illuminate the entire lock in one go.

The lock is rinsed and then washed with shampoo.

D. Results—Comparison of the Shades

The color of the locks was evaluated in the CIE L* a* b* system, using a Minolta Spectrophotometer CM2600D colorimeter. In this L* a* b* system, the three parameters respectively denote the intensity of the color (L*), the green/red color axis (a*) and the blue/yellow color axis (b*).

In this table, the colorimetric parameters L*, a* and b* are given for a lock of untreated hair (TL4), a lock of hair (TL4) treated with the process according to the invention and a lock of hair (TL4) treated with the comparative process using composition (C1).

TABLE 5

| | L* | a* | b* |
|---|---|---|---|
| TL4 chestnut brown lock (untreated) | 21.2 | 3.11 | 4.26 |
| TL4 chestnut brown lock treated with | 33.22 | 8.1 | 15.11 |
| composition C + 405 nm irradiation | | | |
| (invention) | | | |
| TL4 chestnut brown lock treated with | 29.17 | 7.13 | 11.72 |
| composition C1 + 405 nm irradiation | | | |
| (comparative) | | | |

It is observed that for the same amount of active agent used, better bleaching is obtained with the process according to the invention than with the comparative process.

### Example 3

A. Compositions Tested:

Compositions (A8) and (B) from example 1 are used.

B. Light-Emitting Source

The same light-emitting source is used as in example 1.

C. Procedure

1) Process 1

Compositions (A8) and (B) are mixed in a 1:3 ratio in order to obtain an oxidizing composition. The oxidizing composition is then applied to locks of chestnut brown hair (TL4) in a proportion of 10 grams of oxidizing composition per 1 gram of lock. The locks are then enclosed in wrappers that are transparent to the irradiation wavelength and are immediately irradiated for a period of 160 seconds at a fluence of 80 J/cm². The locks of hair are then rinsed.

2) Process 2 (Conventional Bleaching Process with Persalts)

The composition entitled “Infinie Platine Low odor” from L'Oréal Professionnel is mixed with a 30-volumes oxidizing agent (Développeur Oxydant Crème 30 volumes from L'Oréal Professionnel) in a 1:1.5 ratio. The composition obtained from the mixture is applied to locks of chestnut brown hair (TL4) in a proportion of 10 grams of composition per 1 gram of lock. The composition is left on the lock for a period of 40 minutes at 33° C. The locks of hair are then rinsed.

D. Results—Comparison of the Shades

The color of the locks was evaluated in the CIE L* a* b* system, using a Minolta Spectrophotometer CM2600D colorimeter. In this L* a* b* system, the three parameters respectively denote the intensity of the color (L*), the green/red color axis (a*) and the blue/yellow color axis (b*).

In the table below, the colorimetric parameters L*, a* and b* are given for untreated locks of hair (TL4), locks of hair treated with the process according to the prior art (Process 2) and locks of hair treated with the process according to the invention (Process 1). Furthermore, the values Δa* and Δb* are respectively calculated between a*, once the locks have been bleached, and a₀* for the untreated locks, and between b*, once the locks have been bleached, and b₀* for the untreated locks.

TABLE 6

| | L* | a* | b* | Δa* | Δb* |
|---|---|---|---|---|---|
| TL4 chestnut brown | 21.2 | 3.11 | 4.26 | — | — |
| lock (untreated) | | | | | |
| Process 1 (invention) | 54.08 | 5.83 | 19.25 | 2.72 | 14.99 |
| Process 2 (prior art) | 53.5 | 9.5 | 30.06 | 6.39 | 25.8 |

It is observed that for the same degree of bleaching (L), the bleaching processes according to the invention (Process 1) results in lightening that adheres more to the natural tone level of the hair without producing a yellow/orangey lightening background than the process of the prior art.

The values of a* and b* obtained with the process according to the invention are in fact less removed from the initial values a₀* and b₀* for the TL4 untreated locks than the values of a* and b* obtained with the process according to the prior art. Consequently, the locks bleached with process 2 are more red (a*) and more yellow (b*) than those bleached with the process according to the invention.

### Example 4

A. Compositions Tested

The following oxidizing compositions are prepared, the amounts of which are expressed as weight percentages of active material.

TABLE 7

| | Oxidizing | Oxidizing |
|---|---|---|
| | composition D | composition D1 |
| | (invention) | (comparative) |
| Hydrogen peroxide | 7.2 | 7.2 |
| Ammonium carbonate | 1.12 | — |
| Ammonium hydroxide | — | 0.7 |
| Phosphoric acid | — | qs pH 8.9 |
| Water | qs 100 | qs 100 |
| pH | 8.9 | 8.9 |

The source of ammonium is in an equimolar amount in the two compositions.

B. Light-Emitting Source

The same light-emitting source is used as in example 1.

C. Procedure

The compositions (D) and (D1) are applied respectively to locks of hair having a TL4 tone level, in a proportion of 10 grams per 1 gram of lock of hair, and they are enclosed in a wrapper that is transparent to the irradiation wavelength.

Each lock of hair is irradiated at a fluence of 80 J/cm² and at a wavelength of 405 nanometers for a duration of 160 seconds. The light source is moved periodically along the lock so as to never illuminate the entire lock in one go.

The lock is rinsed and then washed with shampoo.

D. Results—Comparison of the Shades

The color of the locks was evaluated in the CIE L* a* b* system, using a

Minolta Spectrophotometer CM2600D colorimeter. In this L* a* b* system, the three parameters respectively denote the intensity of the color (L*), the green/red color axis (a*) and the blue/yellow color axis (b*).

In this table, the colorimetric parameters L*, a* and b* are given for a lock of untreated hair (TL4), a lock of hair (TL4) treated with the process according to the invention using composition (D) and a lock of hair (TL4) treated with the comparative process using composition (D1).

TABLE 8

| | L* | a* | b* |
|---|---|---|---|
| TL4 chestnut brown lock (untreated) | 23.52 | 3.34 | 5.22 |
| TL4 chestnut brown lock treated with | 38.09 | 8.13 | 16.86 |
| composition D + 405 nm irradiation | | | |
| (invention) | | | |
| TL4 chestnut brown lock treated with | 33.56 | 8.45 | 15.90 |
| composition D1 + 405 nm irradiation | | | |
| (comparative) | | | |

It is observed that for the same amount of active agent used, better bleaching is obtained with the process according to the invention than with the comparative process.

1-21. (canceled)

22. A process for bleaching keratin fibers, comprising:
(i) first, applying to said fibers an oxidizing composition comprising:
(a) at least one non-peroxygenated ammonium salt with a molar mass greater than 40 g/mol;
(b) hydrogen peroxide; and
(c) optionally at least one alkaline agent different from the at least one non-peroxygenated ammonium salt with a molar mass greater than 40 g/mol;
the oxidizing composition having a pH greater than or equal to 7.5; and
(ii) second, irradiating said fibers with UV-visible radiation having a wavelength ranging from 200 nm to 800 nm and a fluence ranging from 1 J/cm² to 5000 J/cm².

23. The process of claim 22, wherein the irradiation step is carried out by means of one or more light sources chosen from light-emitting diodes or organic light-emitting diodes.

24. The process of claim 22, wherein after the oxidizing composition is applied to the keratin fibers, the fibers are irradiated for a period of time ranging from 1 minute to 60 minutes.

25. The process of claim 22, wherein the UV-visible radiation has a wavelength ranging from 280 nm to 700 nm.

26. The process of claim 22, wherein the UV-visible radiation has a fluence ranging from 1 J/cm² to 2000 J/cm².

27. The process of claim 22, wherein the at least one non-peroxygenated ammonium salt (a) is chosen from organic ammonium salts.

28. The process of claim 22, wherein the at least one non-peroxygenated ammonium salt (a) is chosen from ammonium carbonate, ammonium bicarbonate, ammonium carbamate, ammonium chloride, ammonium sulfate, ammonium hydrogen sulfate, ammonium nitrate, ammonium phosphate, ammonium monohydrogen phosphate, ammonium dihydrogen phosphate, ammonium lactate, ammonium acetate, or mixtures thereof.

29. The process of claim 22, wherein the at least one non-peroxygenated ammonium salt (a) is chosen from mineral ammonium salts.

30. The process of claim 22, wherein the at least one non-peroxygenated ammonium salt (a) is chosen from acid non-peroxygenated mineral ammonium salts.

31. The process of claim 22, wherein the at least one non-peroxygenated ammonium salt (a) is chosen from alkaline non-peroxygenated mineral ammonium salts.

32. The process of claim 22, wherein the total amount of non-peroxygenated ammonium salts (a) in the oxidizing ranges from 0.1% to 20% by weight, relative to the total weight of the oxidizing composition.

33. The process of claim 22, wherein the oxidizing composition comprises at least one alkaline agent (c) chosen from aqueous ammonia, organic amines, or mixtures thereof.

34. The process of claim 22, wherein the total amount of alkaline agents (c) in the oxidizing composition ranges from 0.1% to 20% by weight, relative to the total weight of the oxidizing composition.

35. The process of claim 22, wherein the total amount of hydrogen peroxide (b) in the oxidizing composition ranges from 0.5% to 12% by weight, relative to the total weight of the oxidizing composition.

36. The process of claim 22, wherein the oxidizing composition comprises less than 5% by weight of persalts, relative to the total weight of the composition.

37. The process of claim 22, wherein the oxidizing composition further comprises one or more hair dyes.

38. The process of claim 22, wherein the oxidizing composition has a pH ranging from 7.5 to 12.

39. An oxidizing composition for bleaching keratin fibers, comprising:
(a) at least one non-peroxygenated ammonium salt with a molar mass greater than 40 g/mol;
(b) hydrogen peroxide; and
(c) optionally at least one alkaline agent different from the at least one non-peroxygenated ammonium salt with a molar mass greater than 40 g/mol,
wherein the oxidizing composition has a pH greater than or equal to 7.5.

40. A process for bleaching keratin fibers, comprising:
mixing:
a first composition (A) comprising at least one non-peroxygenated ammonium salt with a molar mass greater than 40 g/mol and optionally at least one alkaline agent different from the at least one non-peroxygenated ammonium salt with a molar mass greater than 40 g/mol,
a second composition (B) comprising hydrogen peroxide, and
optionally, a third composition comprising at least one alkaline agent different from the at least one non-peroxygenated ammonium salt with a molar mass greater than 40 g/mol;
applying the mixture to the keratin fibers; and
subsequently irradiating said keratin fibers with UV-visible radiation having a wavelength ranging from 200 nm to 800 nm and a fluence ranging from 1 J/cm² to 5000 J/cm².

41. The process of claim 40, wherein the first composition (A) is anhydrous, and the second composition (B) is aqueous.
